(19) United States
(12) Patent Application Publication
(10) Pub. No.: US 2026/0232984 A1
(43) Pub. Date: Aug. 13, 2026

(54) MEDICAL DEVICE FLOW VALVES, AND RELATED SYSTEMS AND METHODS

(71) Applicant: Merit Medical Systems, Inc., South Jordan, UT (US)

(72) Inventor: Michael Dean Haslam, Sandy, UT (US)

(21) Appl. No.: 19/534,419

(22) Filed: Feb. 9, 2026

Related U.S. Application Data
(60) Provisional application No. 63/917,950, filed on Nov. 14, 2025.
(60) Provisional application No. 63/756,608, filed on Feb. 10, 2025.

(51) Int. Cl. A61M 39/28; A61M 39/22

(52) U.S. Cl. CPC A61M 39/283; A61M 2039/226; A61M 2207/00

(57) ABSTRACT

Medical device flow valves are disclosed herein. A medical device flow valve may include a body defining a first bore extending therethrough, a resilient tube disposed within the first bore of the body and defining a lumen, a neck extending from the body and defining a second bore extending therethrough, and a cap secured to the neck and having a plunger positioned at least partially within the second bore of the neck. The cap is selectively rotatable on the neck effective to selectively adjust the cap between (1) an open position in which the lumen of the resilient tube is substantially uniform and undisrupted by the plunger of the cap and (2) a closed position in which the plunger presses against the resilient tube and substantially closes the lumen of the resilient tube.

### RELATED APPLICATIONS

This application claims priority to United States Provisional Application No. 63/756,608, filed on February 10, 2025 and titled, “Medical Device Flow Valves,” and United States Provisional Application No. 63/917,950, filed on November 14, 2025 and titled, “Medical Device Flow Vales, and Related Systems and Methods,” both of which are hereby incorporated by reference in their entireties.

### TECHNICAL FIELD

This application generally relates to medical devices. More particularly, this application relates to medical device flow valves for angiography and other high-pressure medical device applications.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of a flow valve, according to an embodiment.

FIG. 2A is a front cross-sectional view of the flow valve of FIG. 1 in an open position.

FIG. 2B is a front cross-sectional view of the flow valve of FIG. 1 in an intermediate position.

FIG. 2C is a front cross-sectional view of the flow valve of FIG. 1 in a closed position.

FIG. 3 is a rear isometric view of the flow valve of FIG. 1 with the body of the flow valve being transparent.

FIG. 4 is a bottom isometric view of the cap of the flow valve of FIG. 1.

FIG. 5 is a top view of the flow valve of FIG. 1.

FIG. 6 is front isometric view of the flow valve of FIG. 1 with the cap of the flow valve being transparent.

FIG. 7 is a rear isometric view of the flow valve of FIG. 1.

FIG. 8 is a rear cross-sectional view of the flow valve of FIG. 1.

FIG. 9 is a flow diagram of a method of delivering fluid in a medical device system, according to an embodiment.

FIG. 10 is a flow diagram of a method of manufacturing a medical device flow valve, according to an embodiment.

### DETAILED DESCRIPTION

Disclosed herein are embodiments of medical device flow valves (e.g., flow switches), and related systems and methods. Embodiments of the medical device flow valves disclosed herein may be used with any medical device application that involves liquid flow and requires inline shut off capability. Conventional medical device flow valves are typically limited in capability of size, with an inner diameter of approximately 0.065 inches to about 0.075 inches. Many embodiments of the medical device flow valves disclosed herein may have an inner diameter of about 0.120” or about 9 French, thereby allowing for more fluid and/or larger instruments to pass therethrough.

Moreover, conventional medical device flow valves are typically limited to only a completely open position and a completely closed position. In other words, conventional medical device flow valves typically do not allow a user to adjust the medical flow valves to allow a selected amount of fluid to pass through the flow valve that is less than the full amount of fluid when the flow valve is in the open position. In contrast, embodiments of the medical device flow valves disclosed herein are configured to allow a user to not only adjust the flow valves between an open position in which a first, full amount (e.g., first, full flow rate) of fluid passes through the flow valves and a closed position in which substantially no fluid passes through the flow valves, but also one or more intermediate positions in which the one or more intermediate amounts (e.g., one or more intermediate flow rates) of fluid less than the first, full amount of fluid passing through the flow valve in the open position. In many embodiments, the medical device flow valves disclosed herein allow a user to selectively adjust the flow valves to allow any desired intermediate amount (e.g., any desired intermediate flow rate) of fluid that is less than the first, full amount of fluid passing through the flow valves in the open position. Furthermore, embodiments of the flow valves disclosed herein may be configured to completely close the flow of fluid through the flow valves even when a guidewire is extending through the flow valve. Conventional flow valves, such as stopcock valves, are unable to close completely with a guidewire extending through the valve.

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrases “communication with,” “engaged with,” “connected to,” and “coupled to” are used in their ordinary sense, and are broad enough to refer to any suitable coupling or other form of interaction between two or more entities, including mechanical, electrical, magnetic, electromagnetic, fluid, and thermal interaction. Two components may interact with each other even though they are not in direct contact with each other. For example, two components may be coupled to each other through an intermediate component. The directional terms “proximal” and “distal” are used herein to refer to opposite locations on a component or device. The proximal end of a component or device is defined as the end of the device closest to the practitioner when the device is in normal use by the practitioner. The distal end is the end opposite the proximal end, along the longitudinal direction of the device, or the end farthest from the practitioner during normal use.

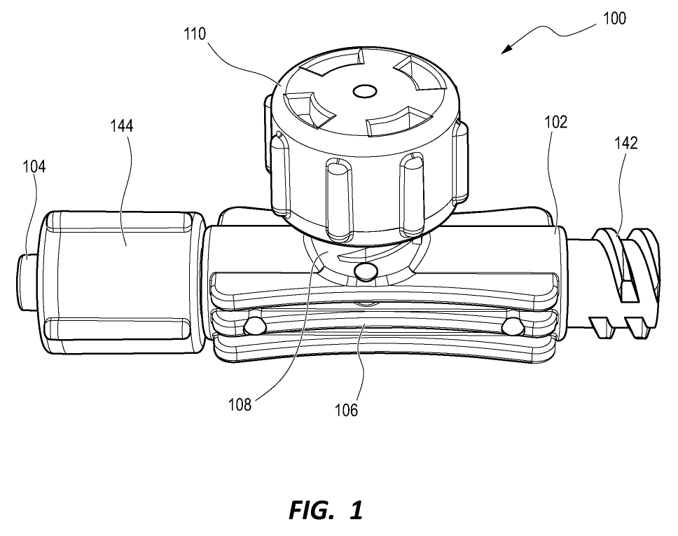

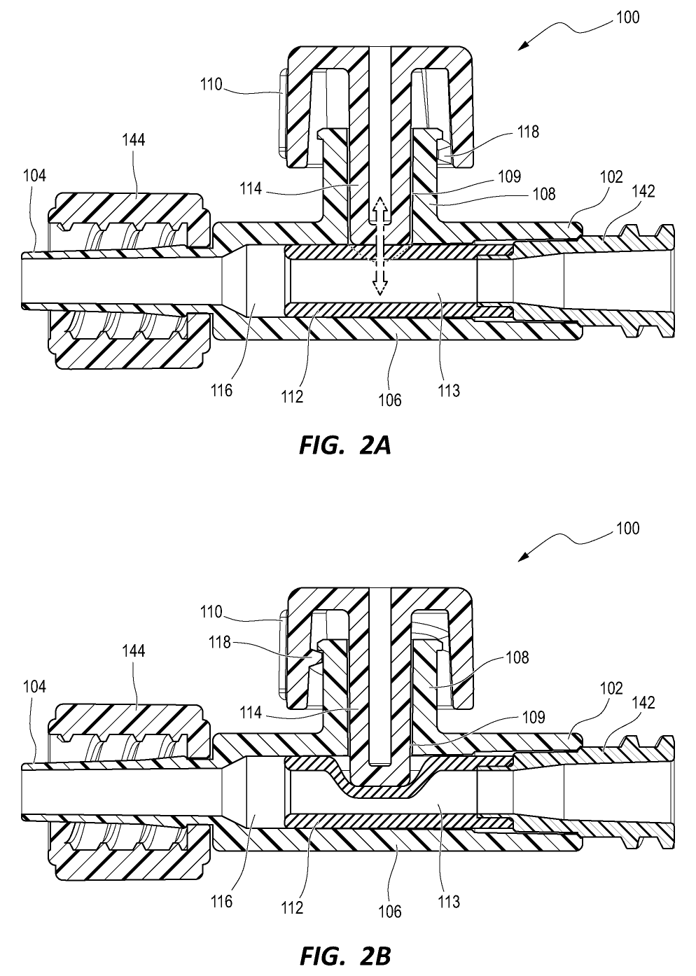

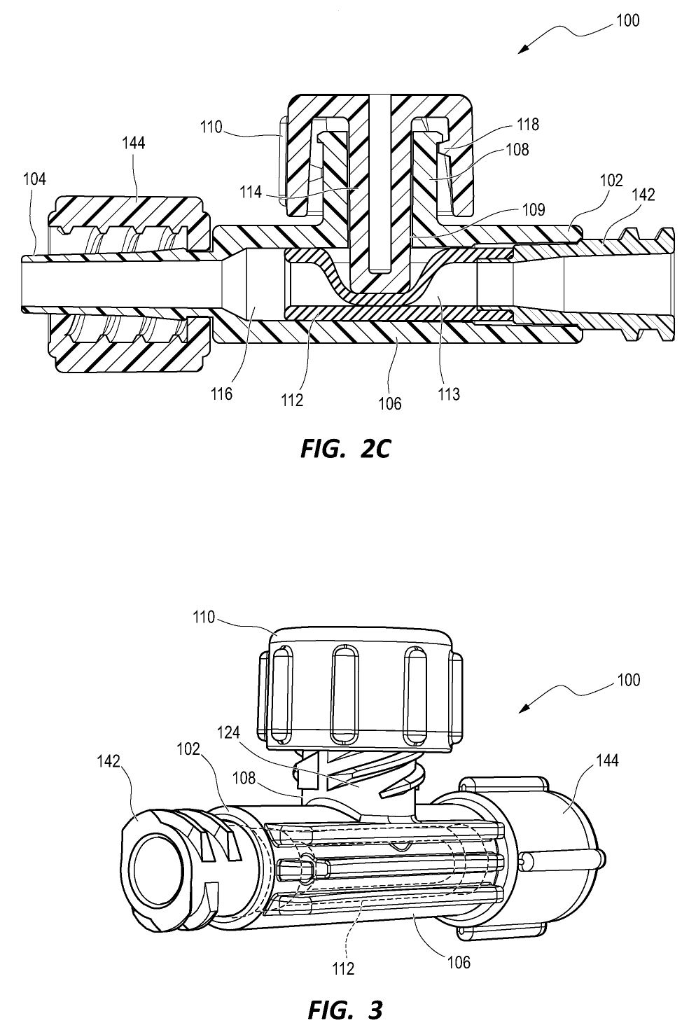

Turning now to the figures, FIG. 1 is a perspective view of a flow valve 100 (e.g., medical device flow valve) and FIGS. 2A-2C are front cross-sectional views of the flow valve 100 in different positions, according to an embodiment. The flow valve 100 may comprise a body 106 defining a first bore 116 extending therethrough, a resilient tube 112 disposed within the first bore 116 of the body 106 and defining a lumen 113, a neck 108 extending from the body and defining a second bore 109 extending therethrough, and a cap 110 secured or securable to the neck 108 and having a plunger 114 positioned or positionable at least partially within the second bore 109 of the neck 108.

The body 106 may be plastic (e.g., polycarbonate) and may comprise a first end region 102 and a second end region 104 having the first bore 116 extending therethrough. In many embodiments, the first end region 102 is configured (e.g., sized and dimensioned) to receive and/or secure to a fastener 142. For example, the flow valve 100 may include a fastener 142 secured or securable to the first end region 102. The fastener 142 may include a female fastener (e.g., female luer fitting) configured to selectively secure to a male fastener (e.g., male luer fitting).

The second end region 104 may include a tapered end region sized and dimensioned to extend through a fastener 144 and/or be inserted into a tube (not shown). For example, in the flow valve 100, a fastener 144 (e.g., male luer fitting) is secured to the second end region 104 of the body 106, with the second end region 104 extending at least partially (e.g., entirely) through the fastener 144. Accordingly, the flow valve 100 may be configured to be secured to tubing inline and be used with any medical device application that involves liquid flow and requires inline shut off capability.

When secured to the neck 108, the cap 110 is selectively rotatable on the neck 108 to move the plunger 114 within the second bore 109. For example, the cap 100 may be rotated (1) in a first direction (e.g., clockwise) to move the plunger 114 into or further into the lumen 116 and (2) in a second direction (e.g., counter-clockwise) to move or pull the plunger 114 at least partially or completely out of the lumen 116. Rotation of the cap 110 to at least partially move the plunger 114 into the first bore 116 pinches and at least partially or completely closes the resilient tube 112 positioned within the first bore 116. Accordingly, the cap 110 may be selectively rotatable on the neck 108 effective to selectively adjust the cap 110 between (1) an open position (shown in FIG. 2A) in which the lumen 113 of the resilient tube 112 is substantially uniform and undisrupted (e.g., completely open) by the plunger 114 of the cap 110 and (2) a closed position (shown in FIG. 2C) in which the plunger 114 presses against the resilient tube 112 and substantially closes the lumen 113 of the resilient tube 112. In some embodiments, the end of the plunger 114 that is at least proximate (e.g., interfacing or adjacent) to the resilient tube 112 is substantially planar or flat.

Said another way, the cap 110 is selectively rotatable on the neck 108 effective to allow a user to selectively adjust the cap 110 between (1) the open position shown in FIG. 2A in which the plunger 110 does not enter the first bore 116 and/or does not decrease a diameter of the lumen 113 of the resilient tube 112 and (2) the closed position shown in FIG. 2C in which the plunger 110 enters the first bore 116 and presses against the resilient tube 112 sufficient to substantially close the lumen 113 of the resilient tube 112. Accordingly, when the cap 110 is in the open position, the flow rate or amount of fluid passing through the lumen 113 of the resilient tube 112 (and the flow valve 100 generally) is unaffected by the cap 110 and the plunger 110 and is a full flow rate or full amount of fluid. When the cap 110 is in the closed position, the flow rate or amount of fluid passing through the lumen 113 of the resilient tube 112 (and the flow valve 100 generally) is substantially blocked by the plunger 114 pinching or pressing against resilient tube 112 to close the lumen 113 such that the flow or amount of fluid passing through the lumen 113 is substantially zero.

In use, the flow valve 100 may sometimes be used with a guidewire (not shown) extending through the first bore 116 of the body 106 and the lumen 113 of the resilient tube 112. Convention flow valves do not allow the flow of fluid through the flow valve to be completely or substantially stopped when a guidewire is extending through the flow valve. In many embodiments of the flow valve 100 disclosed herein, however, the cap 110 is selectively rotatable on the neck 108 effective to selectively adjust the cap 110 to the closed position shown in FIG. 2B in which the plunger 114 presses against the resilient tube 112 and substantially closes the lumen 113 of the resilient tube 112 with a guidewire (not shown) extending through the lumen 113 of the resilient tube 112.

In many embodiments of the flow valve 100 the cap 110 is further selectively rotatable on the neck 108 effective to selectively adjust the cap 110 to one or more intermediate positions (shown in FIG. 2B) in which the plunger 114 presses against the resilient tube 112 and only partially closes the lumen 113 of the resilient tube 112. For example, the cap 110 may be rotated to any position between the open position and the closed position, and thereby decreases the diameter of the lumen 113 and the flow rate or amount of fluid passing through the lumen 113 of the resilient tube 112 (and the flow valve 100 generally). Accordingly, a user may select any flow rate or amount of fluid between the full flow rate in the open position and the closed lumen 113 or flow valve 100 (e.g., zero flow rate) by rotating the cap 110 to any position between the open position and the closed position. This feature allows a user to find a desired flow rate between the full flow rate and the closed flow rate when the user desires some fluid to flow through the flow valve 100 but not the full flow rate.

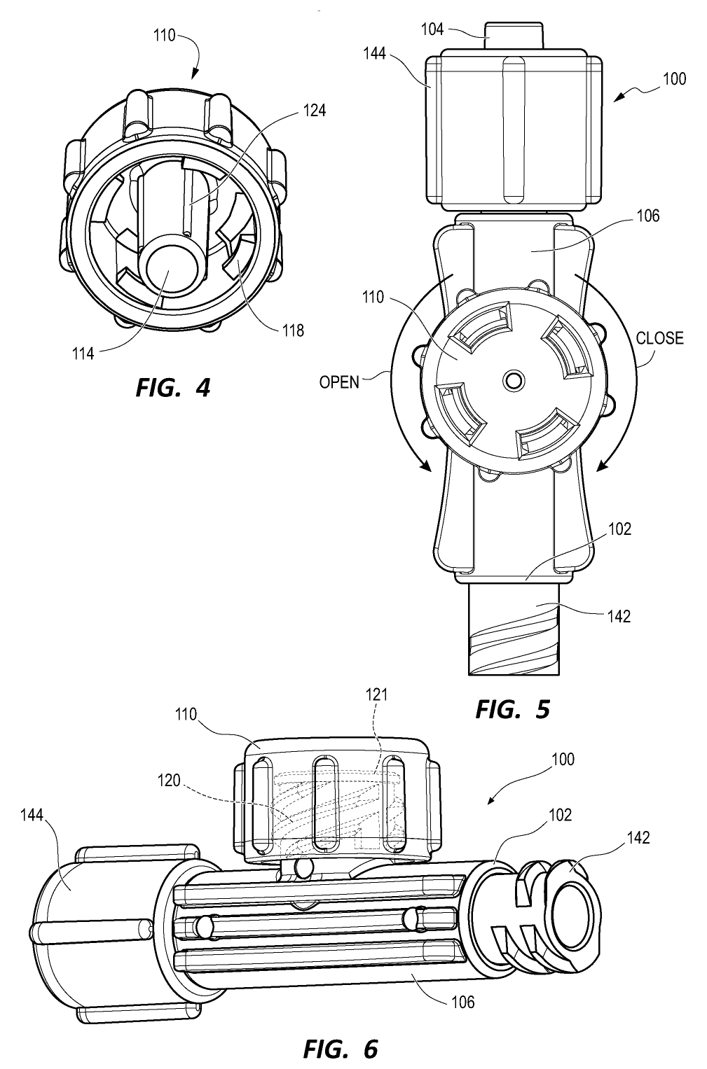

Turning ahead in the drawings, FIG. 3 is a rear isometric view of the flow valve 100 of FIG. 1 with the body 106 of the flow valve 100 being transparent, and FIG. 4 is a bottom isometric view of the cap 110 of the flow valve of FIG. 1. In many embodiments, the plunger 114 comprises one or more ribs 124 (shown in FIG. 4) configured to provide an interference fit with the inner diameter of the neck 108 in the second bore 109. For example, the plunger 114 may comprise a plurality of ribs 124 (e.g., two, three, four, or more ribs) protruding from an outer surface of the plunger 114. The plurality of ribs 124 are configured to contact the inner diameter of the neck 108 defining the second bore 109 effective to retain the cap 110 at the one or more intermediate positions (e.g., any intermediate position) between the open position and the closed position. Thus, a user may find a desired flow between the full flow rate and the closed position and the cap 110 by rotating the cap 110 at any position between the open position and the closed position, and the ribs 124 may help retain the cap 110 in the desired position allowing the desired flow rate through the flow valve 100. In some embodiments, the neck 108 may include one or more ribs projecting inward into the second bore 109 and configured engage with the plurality of ribs 124 to retain the cap 110 in the one or more intermediate positions, while also allowing the plurality of ribs 124 to be rotated over the one or more ribs of the neck as the cap 110 is rotated clockwise or counter-clockwise.

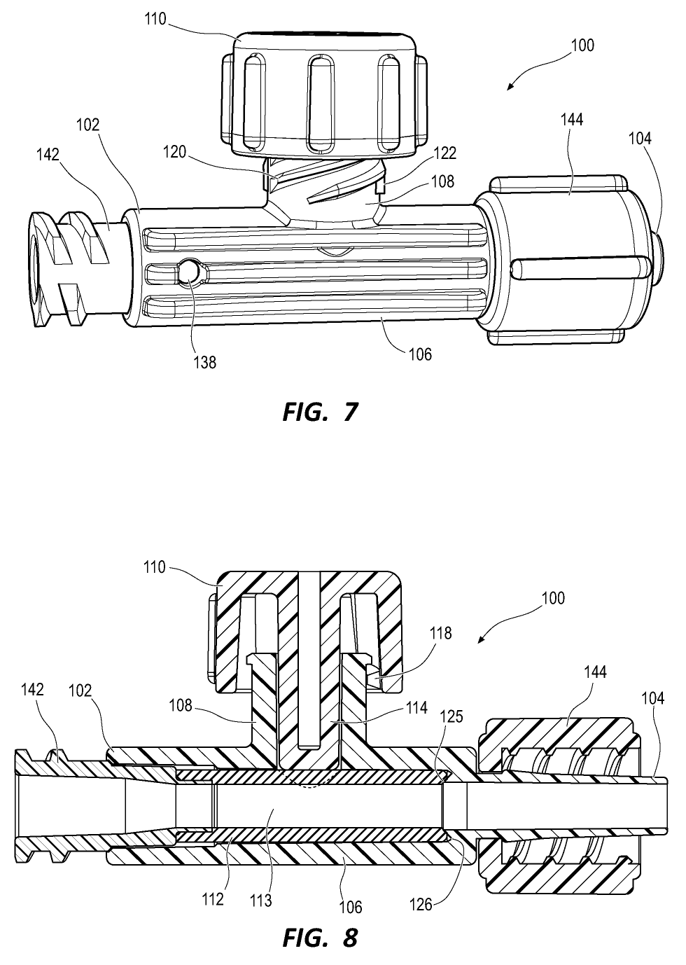

Turning ahead in the drawings, FIG. 5 is a top view of the flow valve 100, FIG. 6 is front isometric view of the flow valve 100 with the cap 110 of the flow valve 100 being transparent, and FIG. 7 is a rear isometric view of the flow valve 100. In many embodiments, the cap 110 may be rotated a first direction (e.g., clockwise) to partially or completely close the flow valve 100 and rotated a second direction (e.g., counterclockwise) to partially or completely open the flow valve 100. For example, the cap 110 may be threadedly coupled to the neck 108. Accordingly, the cap 110 may comprise one or more threads or lugs 118 (shown in FIG. 4) and the neck 108 may comprise one or more threads 120 on an outer diameter of the neck 108 configured to threadedly engage with one or more threads or lugs 118 of the cap 110. The one or more threads 120 and/or the one or more lugs 118 may be positioned at a selected pitch for a desired amount of rotation of the cap 110 between the open position and the closed position. In a particular embodiment, the flow valve 100 is configured such that the cap 110 is rotated about ½ to about ¾ between the open position and the closed position (with the cap 110 being configured to be retained at any rotation position between the open position and the closed position for a partially open or partially closed position). In some embodiments, the flow valve 100 is configured such that the cap 110 is rotated about ¾ to about 1¼ between the open position and the closed position (with the cap 110 being configured to be retained at any rotation position between the open position and the closed position for a partially open or partially closed position).

In many embodiments, the one or more lugs 118 comprises a plurality of lugs 118 (e.g., discontinuous lugs that do not extend entirely around the cap 110). For example, the cap 110 may comprise a plurality of lugs 118 disposed substantially equal radial distance from adjacent lugs of the plurality of lugs 118. In the embodiment shown in FIG. 4, the cap 110 comprises four lugs 118 disposed approximate 90 degrees from adjacent lugs of the plurality of lugs 118. In other embodiments, the cap 110 may comprise two, three, or more lugs 118. In many embodiments, the neck 108 comprises a flanged end 121 opposite to the body 106. The one or more lugs 118 may be configured to snap over the flanged end 121 of the neck when securing the cap 110 to the neck 108 and prevent or inhibit the cap 110 from rotating off the neck 108. In some embodiments, the flanged end 121 is chamfered to allow for easier positioning of the cap 110 onto the neck 108 while still inhibiting or preventing removal or rotation of the cap 110 off the neck 108.

In many embodiments, the flow valve 100 may be configured to provide an indication to the user that the cap 110 is in the fully closed position and/or provide a stronger engagement or resistance to rotation when the cap 110 is in the fully closed position. For example, the neck 108 may comprise a detent 122 or a flat configured to retain the cap 110 in the closed position by engaging at least one lug of the one or more lugs 118. As provided above, many embodiments comprise a plurality of lugs 118. As the plurality of lugs 118 are spaced from adjacent lugs of the plurality of lugs 118, one lug of the plurality of lugs 118 may engage the detent 122 or flat to retain the cap 110 in the closed position. When rotating the cap 110 to the closed position, the cap 110 may click when a lug of the plurality of lugs 118 rotates over the detent 122 to engage the lug with the detent 122, indicating to the user that the flow valve 100 is in the closed position. The detent 122 also may provide increase friction or resistance to rotation for retaining the flow valve 100 in the closed position.

In some embodiments, the body 106 defines a hole 138 positioned to receive an adhesive for securing at least one of the first fastener 142 or the second fastener 144 to the body 106. For example, the body 106 may define a hole 138 positioned at least proximate to the first end region 102 and configured to adhesively secure the first fastener 142 to the first end region 102 of the body 106.

Turning ahead in the drawings, FIG. 8 is a rear cross-sectional view of the flow valve 100 illustrating the resilient tube 112 positioned within the first bore 116 of the body 106. The resilient tube 112 may be formed of a resilient material, such as a silicone, having a softness that allows compression of the resilient tube 112 by the plunger 114 but also returns to its original (substantially cylindrical) shape when the plunger 114 is removed from pressing against the resilient tube 112. In many embodiments, the resilient tube 112 includes a material that also provides resistance against rotation of the cap 110 from the open position to the closed position effect to promote retention of the cap 110 at any position between the open position and the closed position. The resilient tube 110, for example, may comprise a shore A durometer of less than about 50, such as a shore A of about 35 to about 40. In some embodiments, the resilient tube 112 is injection molded (e.g., liquid silicone rubber (LSR) injection molded) or extruded.

The lumen 113 of the resilient tube 112 also may include a diameter or size greater than conventional flow valves. For example, the lumen 113 of the resilient tube may be greater than about 0.1 inch, such about 0.1 to about 0.2, about 0.110 to about 0.130, or about 0.115 to about 0.125. In some embodiments, the lumen 113 may be sized to accommodate 9 French instruments.

In some embodiments, the body 106 includes an interior ledge 125 within the first bore 116 and having an annular recess 126. At least a portion of an end region of the resilient tube 112 may be seated within the annular recess 126 in the interior ledge 125. With at least a portion of the end region of the resilient tube 112 being seated within the annular recess 126, the interior ledge 125 keeps the end of the resilient tube 112 open (e.g., prevents or inhibits the end region of the resilient tube 112 from closing). In some embodiments, the end region of the resilient tube 112 is molded into the annular recess 126 of the interior ridge 125. In some embodiments, the end region of the resilient tube 112 is shaped complementary to the annular recess 126 such that the end region of the resilient tube 112 mates with the interior ridge 125 in the annular recess 126.

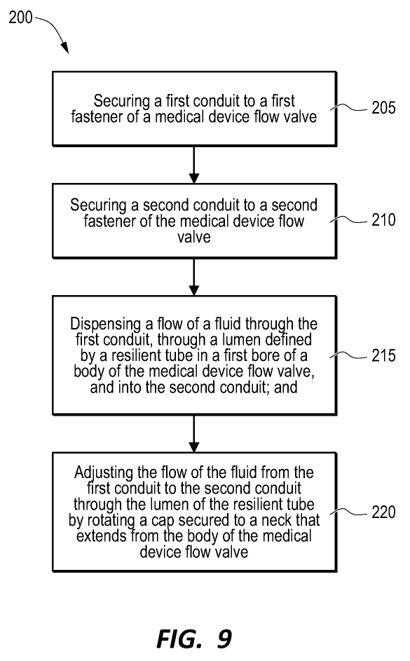

Also disclosed herein are methods of adjusting a flow of fluid in a medical device system. The methods may comprise adjusting the cap 110 on any of the medical device flow valves 100 disclosed herein. FIG. 9, for example, is a flow diagram of a method 200 of delivering fluid in a medical device system, according to an embodiment. The method 200 may comprise securing 205 a first conduit to a first fastener of a medical device flow valve and securing 210 a second conduit to a second fastener of the medical device flow valve. The method 200 also may comprise dispensing 215 a flow of a fluid through the first conduit, through a lumen defined by a resilient tube in a first bore of a body of the medical device flow valve, and into the second conduit. The method also may comprise adjusting 220 the flow of the fluid from the first conduit to the second conduit through the lumen of the resilient tube by rotating a cap secured to a neck that extends from the body of the medical device flow valve. The neck may define a second bore extending therethrough. In adjusting 220 the flow of the fluid from the first conduit to the second conduit through the lumen of the resilient tube, the cap may be rotated between at least one of (1) an open position in which the lumen of the resilient tube is substantially uniform and undisrupted by a plunger of the cap in the second bore of the neck, (2) one or more intermediate positions in which the plunger presses against the resilient tube and only partially closes the lumen of the resilient tube, or (3) a closed position in which the plunger presses against the resilient tube and substantially closes the lumen of the resilient tube to adjust the flow of the fluid from the first conduit to the second conduit.

In some embodiments of the method 200, adjusting 220 the flow of the fluid may comprise at least adjusting the flow of the fluid by rotating the cap to the one or more intermediate positions and retaining the cap at the one or more intermediate positions with one or more ribs on the neck that provide an interference fit with the neck in the second bore to retain the cap at the one or more intermediate positions between the open position and the closed position that only partially close the lumen of the resilient tube.

The cap may be threadedly coupled to the neck, and adjusting 220 the flow of the fluid may comprise at least rotating the cap to the closed position and retaining the cap in the closed position with a detent or a flat on the neck that retains the cap in the closed position with the plunger pressing against the resilient tube to substantially close the lumen of the resilient tube.

In many embodiments of the method 200, the lumen of the resilient tube is at least about 9 French and/or has a diameter of about 0.120 inch. The resilient tube may comprise a silicone material having a shore A durometer of about 50 or less. The body also may define a hole positioned to receive an adhesive for securing at least one of the first fastener or the second fastener to the body. In some embodiments of the method 200, the body includes an interior ledge within the first bore and having an annular recess. At least a portion of an end region of the resilient tube may be seated within the annular recess. The neck may comprise a flanged end and the cap comprises a plurality of lugs configured to snap over the flanged end of the neck and prevent the cap from rotating off the neck. In some embodiments, the neck may comprise a detent or a flat configured to engage with at least one lug of the plurality of lugs and retain the cap in the closed position. The cap in the method 200 may be selectively rotatable on the neck effective to selectively adjust the cap to the closed position in which the plunger presses against the resilient tube and substantially closes the lumen of the resilient tube with a guidewire extending through the lumen of the resilient tube.

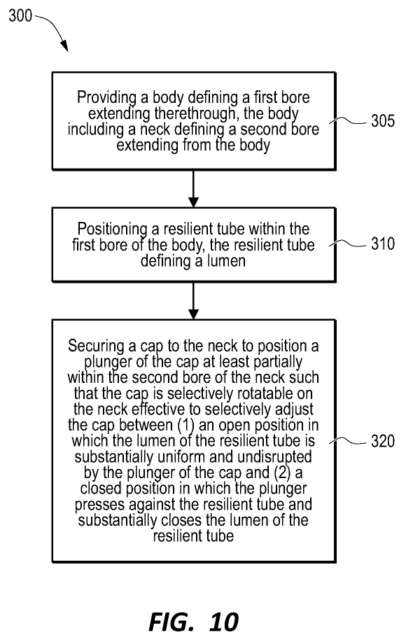

Also disclosed herein are methods of manufacturing a medical device flow valve. The methods may comprise manufacturing of any of the medical device flow valves 100 disclosed herein. FIG. 10, for example, is a flow diagram of a method 300 of manufacturing a medical device flow valve, according to an embodiment. The method 300 may comprise providing 305 a body defining a first bore extending therethrough, the body including a neck defining a second bore extending from the body. The method 300 also may comprise positioning 310 a resilient tube within the first bore of the body, the resilient tube defining a lumen. The method 300 also may comprise securing 320 a cap to the neck to position a plunger of the cap at least partially within the second bore of the neck such that the cap is selectively rotatable on the neck effective to selectively adjust the cap between (1) an open position in which the lumen of the resilient tube is substantially uniform and undisrupted by the plunger of the cap and (2) a closed position in which the plunger presses against the resilient tube and substantially closes the lumen of the resilient tube.

In some embodiments of the method 300, the cap is further selectively rotatable on the neck effective to selectively adjust the cap to one or more intermediate positions in which the plunger presses against the resilient tube and only partially closes the lumen of the resilient tube. Moreover, the plunger formed according to the method 300 may comprise one or more ribs configured to provide an interference fit with the neck in the second bore and retain the cap at the one or more intermediate positions between the open position and the closed position.

In some embodiments of the method 300, securing 320 the cap to the neck comprises threadedly coupling the cap to the neck. The neck may comprise a detent or a flat configured to retain the cap in the closed position. The lumen of the resilient tube in the method 300 may be at least about 9 French and/or have a diameter of about 0.120 inch. Moreover, the resilient tube may comprise a silicone material having a shore A durometer of about 50 or less.

The method 300 also may comprise securing a first fastener to a proximal end region of the body and securing a second fastener secured to a distal end region of the body. The method 300 also may comprise inserting an adhesive through a hole in the body to secure at least one of the first fastener or the second fastener to the body.

In some embodiments of the method 300, the body includes an interior ledge within the first bore and having an annular recess. Positioning 310 the resilient tube within the first bore of the body may then comprise seating at least a portion of an end region of the resilient tube being within the annular recess. The neck in the method 300 may comprise a flanged end and the cap may comprise a plurality of lugs configured to snap over the flanged end of the neck and prevent the cap from rotating off the neck. The neck also may comprise a detent or a flat configured to engage with at least one lug of the plurality of lugs and retain the cap in the closed position. The medical flow device formed according to the method 300 also may include the cap being selectively rotatable on the neck effective to selectively adjust the cap to the closed position in which the plunger presses against the resilient tube and substantially closes the lumen of the resilient tube with a guidewire extending through the lumen of the resilient tube.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, only a portion of a method described herein may be a separate method. Stated otherwise, some methods may include only a portion of the steps described in a more detailed method.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof, for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of this disclosure.

1. A medical device flow valve, comprising:
a body defining a first bore extending therethrough;
a resilient tube disposed within the first bore of the body and defining a lumen;
a neck extending from the body and defining a second bore extending therethrough; and
a cap secured to the neck and having a plunger positioned at least partially within the second bore of the neck, wherein the cap is selectively rotatable on the neck effective to selectively adjust the cap between (1) an open position in which the lumen of the resilient tube is substantially uniform and undisrupted by the plunger of the cap and (2) a closed position in which the plunger presses against the resilient tube and substantially closes the lumen of the resilient tube.

2. The medical device flow valve of claim 1, wherein the cap is further selectively rotatable on the neck effective to selectively adjust the cap to one or more intermediate positions in which the plunger presses against the resilient tube and only partially closes the lumen of the resilient tube.

3. The medical device flow valve of claim 2, wherein the plunger comprises one or more ribs configured to provide an interference fit with the neck in the second bore and retain the cap at the one or more intermediate positions between the open position and the closed position.

4. The medical device flow valve of claim 1, wherein the cap is threadedly coupled to the neck.

5. The medical device flow valve of claim 4, wherein the neck includes a detent or a flat configured to retain the cap in the closed position.

6. The medical device flow valve of claim 1, further comprising a first fastener secured to a proximal end region of the body and a second fastener secured to a distal end region of the body.

7. The medical device flow valve of claim 6, wherein the body defines a hole positioned to receive an adhesive for securing at least one of the first fastener or the second fastener to the body.

8. The medical device flow valve of claim 1, wherein the body includes an interior ledge within the first bore and having an annular recess, at least a portion of an end region of the resilient tube being seated within the annular recess.

9. The medical device flow valve of claim 1, wherein the neck comprises a flanged end and the cap comprises a plurality of lugs configured to snap over the flanged end of the neck and prevent the cap from rotating off the neck.

10. The medical device flow valve of claim 9, wherein the neck includes a detent or a flat configured to engage with at least one lug of the plurality of lugs and retain the cap in the closed position.

11. The medical device flow valve of claim 1, wherein the cap is selectively rotatable on the neck effective to selectively adjust the cap to the closed position in which the plunger presses against the resilient tube and substantially closes the lumen of the resilient tube with a guidewire extending through the lumen of the resilient tube.

12. A method of delivering fluid in a medical device system, the method comprising:
securing a first conduit to a first fastener of a medical device flow valve;
securing a second conduit to a second fastener of the medical device flow valve;
dispensing a flow of a fluid through the first conduit, through a lumen defined by a resilient tube in a first bore of a body of the medical device flow valve, and into the second conduit; and
adjusting the flow of the fluid from the first conduit to the second conduit through the lumen of the resilient tube by rotating a cap secured to a neck that extends from the body of the medical device flow valve, the neck defining a second bore extending therethrough, wherein the cap including a plunger positioned at least partially within the second bore of the neck and the cap is rotated between at least one of (1) an open position in which the lumen of the resilient tube is substantially uniform and undisrupted by the plunger of the cap, (2) one or more intermediate positions in which the plunger presses against the resilient tube and only partially closes the lumen of the resilient tube, or (3) a closed position in which the plunger presses against the resilient tube and substantially closes the lumen of the resilient tube to adjust the flow of the fluid from the first conduit to the second conduit.

13. The method of claim 12, wherein adjusting the flow of the fluid includes at least adjusting the flow of the fluid by rotating the cap to the one or more intermediate positions and retaining the cap at the one or more intermediate positions with one or more ribs on the neck that provide an interference fit with the neck in the second bore to retain the cap at the one or more intermediate positions between the open position and the closed position that only partially close the lumen of the resilient tube.

14. The method of claim 13, wherein the cap is threadedly coupled to the neck, and
wherein adjusting the flow of the fluid includes at least rotating the cap to the closed position and retaining the cap in the closed position with a detent or a flat on the neck that retains the cap in the closed position with the plunger pressing against the resilient tube and substantially closing the lumen of the resilient tube.

15. The method of claim 14, wherein the body defines a hole positioned to receive an adhesive for securing at least one of the first fastener or the second fastener to the body.

16. The method of claim 14, wherein the body includes an interior ledge within the first bore and having an annular recess, at least a portion of an end region of the resilient tube being seated within the annular recess.

17. The method of claim 14, wherein the neck comprises a flanged end and the cap comprises a plurality of lugs configured to snap over the flanged end of the neck and prevent the cap from rotating off the neck.

18. The method of claim 17, wherein the neck includes a detent or a flat configured to engage with at least one lug of the plurality of lugs and retain the cap in the closed position.

19. The method of claim 14, wherein the cap is selectively rotatable on the neck effective to selectively adjust the cap to the closed position in which the plunger presses against the resilient tube and substantially closes the lumen of the resilient tube with a guidewire extending through the lumen of the resilient tube.

20. A method of manufacturing a medical device flow valve, the method comprising:
providing a body defining a first bore extending therethrough, the body including a neck defining a second bore extending from the body;
positioning a resilient tube within the first bore of the body, the resilient tube defining a lumen; and
securing a cap to the neck to position a plunger of the cap at least partially within the second bore of the neck such that the cap is selectively rotatable on the neck effective to selectively adjust the cap between (1) an open position in which the lumen of the resilient tube is substantially uniform and undisrupted by the plunger of the cap and (2) a closed position in which the plunger presses against the resilient tube and substantially closes the lumen of the resilient tube.
